(19) United States
(12) Patent Application Publication
(10) Pub. No.: US 2006/0025739 A1
(43) Pub. Date: Feb. 2, 2006

(54) Wrapper component for personal care articles having a sensory cue for opening

(76) Inventors: Joseph DiPalma, Neenah, WI (US); Adrienne R. Loyd, Neenah, WI (US); Marie A. Mathews, Appleton, WI (US); Kristi Jo Bryant, Appleton, WI (US); Garry Roland Woltman, Greenville, WI (US); Sarah Lois Wilkes, Appleton, WI (US); Sheila Marie Heyrman, Appleton, WI (US); James Joseph Clark JR., Appleton, WI (US); Brian Lee Thomas, Neenah, WI (US); Laura Jane Walker, Appleton, WI (US); Shelley Rae Rasmussen, Oshkosh, WI (US); Steven James Nielsen, Conway, AR (US); Mark Leonard Ingle, Vilonia, AK (US); Richard Lee Rothe, Neenah, WI (US); Richard Joseph Hantke, Appleton, WI (US)

Correspondence Address: KIMBERLY-CLARK WORLDWIDE, INC., 401 NORTH LAKE STREET, NEENAH, WI, 54956, US

(21) Appl. No.: 11/005,684

(22) Filed: Dec. 7, 2004

Related U.S. Application Data
(63) Continuation-in-part of application No. 10/750,424, filed on Dec. 30, 2003.

(51) Int. Cl. A61F 13/15

(52) U.S. Cl. 604/385.02; 604/361

(57) ABSTRACT

The present invention provides a personal care product including a wrapper having at least one free edge, an absorbent personal care article and a sensory cue providing indicia as to the location of the free edge. In one embodiment, the wrapper is a wrapper component and has a first panel, a back panel and a second panel, where the first panel is connected to the back panel and the second panel is connected to the back panel at an opposite end from where the first panel is connected to the back panel. The second panel having a free edge positioned adjacent the first panel, wherein the second panel has a first set of sensory attributes which terminate at the free edge of the second panel and the first panel has a second set of sensory attributes which are adjacent the free edge of the second panel. In the present invention, at least one attribute from the first set of sensory attributes or at least one attribute from the second set of attributes is different from attributes from the other set. The different attribute provides a contrast between the second panel and the first panel, and this contrast provides a sensory cue providing clear indicia as to the location of the free edge of the second panel. Also provided is a method of using the personal care product and wrapper. The sensory cue can also be used to direct a user as to how to open the wrapper.

This application is a continuation-in-part of U.S. patent application Ser. No. 10/750,424, filed Dec. 30, 2003, and claims the benefit of the filing date of U.S. patent application Ser. No. 10/750,424.

### FIELD OF THE INVENTION

The present invention relates generally to a personal care product, and in particular, to a wrapper component for a personal care product that includes a sensory cue directed at a portion of the wrapper component so as to aid the user in opening the wrapper component.

### BACKGROUND OF THE INVENTION

Absorbent articles such as pantiliners, feminine napkins and tampons are frequently carried about in purses, backpacks, briefcases, and the like until needed. Typically, the articles are placed in purses, backpacks, briefcases, and the like such that they are loose, and are free to move within the purses, backpacks, briefcases, and the like with the other items contained therein. Unfortunately, purses, backpacks, briefcases, and the like do not always provide a hygienic environment for the articles, and thus the articles can become dirty and/or damaged. Further, the articles can become scattered about in purses, backpacks, briefcases, and the like so they are difficult to find when needed.

In the past, specifically designed pouches have been distributed for holding several absorbent articles at a time. These pouches reduce contact between the articles and potentially non-hygienic environments, and make the articles easier to find when needed. These pouches are made from durable materials such as heavy vinyl so they can be reused, but reuse necessitates the pouches are refilled from time to time. Further, the pouches frequently become non-hygienic after extended use, requiring them to be cleaned or discarded and replaced.

To avoid the problems described above, often personal care products, including for example feminine pads, tampons and pantiliners, are individually wrapped in a pouch or similar wrapper, which includes a flap that closes the pouch. Typically, such pouches are a solid color, or have a pattern that covers the entirety of the wrapper. As such, it can be difficult for the user to locate the flap to open the pouch, since the flap is not visually distinguishable from the remainder of the wrapper.

To avoid the problem of locating the edge of the flap, pouches in the past have been provided with the phrase “open here” and the like near the end of the flap to direct the user to the location of the pouch opening. However, having words or phrases like “open here” on the flap lowers any aesthetic value the pouch may have. In addition, by placing the phrase in a particular language, those who are unable to read the language used may not be directed to the location of the opening flap on the pouch. The problem of locating the flap can be exacerbated for users with impaired eyesight. Therefore, placing a phrase at the end of the flap on the pouch may not provide a person with a visual impairment with a sensory cue as to the location of the flap and opening.

Other attempts have been used to direct a user's attention to the end of the flap on pouches. These include placing an edge color on the edge of the flap to highlight the edge. Generally, this is done by placing a narrow band of a different color along the edge of the flap, as is shown in FIGS. 10A and 10B. However, given that the color or pattern on the flap above the edge of color is the same as the color on the front panel below the flap, it is often still difficult to distinguish the edge of the flap from the panel. Such an indicator on a flap often requires a user to study the wrapper beyond a first look to ascertain the location of the free end of the flap.

There is a need in the art for a way to clearly convey to a user of absorbent products wrapped in a wrapper component where the opening of the wrapper is located, and how to open the wrapper, while providing an aesthetically pleasing appearance to the wrapper.

### SUMMARY OF THE INVENTION

Generally stated, the present invention provides personal care product having at least one wrapper with at least one free edge; at least one personal care article disposed within the wrapper; and at least one sensory cue providing clear indicia as to the location of at least one free edge of the wrapper.

In other aspect of the present invention, the present invention also provides a personal care product including a wrapper component and an absorbent personal care article. The wrapper has a first panel, a back panel and a second panel, where the first panel is connected to the back panel and the second panel is connected to the back panel at an opposite end from where the first panel is connected to the back panel. The second panel having a free edge positioned adjacent the first panel, wherein the second panel has a first set sensory attributes which terminate at the free edge of the second panel and the first panel has a second set of sensory attributes which are adjacent the free edge of the second panel. In the present invention, at least one attribute from the first set of sensory attributes or at least one attribute from the second set of attributes is different from attributes from the other set. The different attribute provides a contrast between the second panel and the first panel, and this contrast provides a sensory cue providing clear indicia as to the location of the free edge of the second panel. The wrapper component may have more than two panels. In one embodiment, the second panel may be a flap.

Another aspect of the present invention provides an indication to the user as to the location of the free edge and an indication of how to open the wrapper component to access the absorbent article within the wrapper. In this aspect of the present invention, the sensory cue may be configured to mimic other sensory cues commonly found in every day life which tend to indicate an opening and how to open the opening.

The present invention also provides a method of using a personal care product which includes providing a personal care product component placed in a wrapper component, locating a free edge using a sensory cue provided on the wrapper; and manipulating the second panel of the wrapper and opening said wrapper component to access the personal care product component disposed within the wrapper.

### BRIEF DESCRIPTION OF THE DRAWINGS

FIG. 1 is a body-side plan view of an exemplary personal care product component with a portion thereof partially cut away.

FIG. 2A is a perspective view of a wrapper component in an open configuration with a personal care product component in a folded configuration.

FIG. 2B is a perspective view of another wrapper component in an open configuration with a personal care product component in a unfolded configuration.

FIG. 2C is a perspective view of the wrapper component in a partially open configuration with the personal care product component in a partially folded configuration.

FIGS. 3A-3J are plan views of various wrapper components.

FIG. 4 is a schematic cross-section of one embodiment of a wrapper component, with various dimensions and proportions at the ends thereof exaggerated for the sake of illustration.

FIG. 5A is a plan view of one embodiment of a wrapper component material in an unfolded configuration.

FIG. 5B is a plan view of the wrapper component material shown in FIG. 5A in a folded configuration.

FIG. 6 is a plan view of an alternative embodiment of a wrapper component.

FIG. 7 is a plan view of an alternative embodiment of a wrapper component.

FIG. 8 is schematic graphical illustration of hue, luminosity and saturation/vividness.

FIG. 9A is a schematic cross-section of one embodiment of a wrapper component, with various dimensions and proportions at the ends thereof exaggerated for the sake of illustration.

FIG. 9B is a plan view of the embodiment shown in FIG. 9A.

FIG. 9C is a front view of another embodiment of the present invention.

FIGS. 10A and 10B show prior art wrapper components in a closed position.

FIG. 11 shows another embodiment of the present invention which provides a sensory cue of how to open the wrapper component.

FIG. 12 shows another embodiment of the present invention which provides a sensory cue of how to open the wrapper component.

FIGS. 13A, 13B and 13C each show a plan view of one embodiment of a wrapper component material in a folded configuration.

### DEFINITIONS

It should be noted that, when employed in the present disclosure and claims, the terms “comprises”, “comprising” and other derivatives from the root term “comprise” are intended to be open-ended terms that specify the presence of any stated features, elements, integers, steps, or components, and are not intended to preclude the presence or addition of one or more other features, elements, integers, steps, components, or groups thereof.

It should be understood that the term “personal care article” as used herein refers to any article used to control bodily fluids, and includes “absorbent products,” which refers to any article configured to absorb and retain bodily exudates, including urine, bowel movements, blood and menses, and includes such a product in a packaged and unpackaged configuration. As such, personal care products, as used herein, includes without limitation, diapers, child toilet training pants, adult incontinence garments, male incontinence products, tampons, vaginal suppositories, panty liners, pads, sanitary napkins, tissues, wipes, etc. Examples of commercially available personal care products include, without limitation, Poise® feminine care products, including pantiliners and pads, and Kotex® feminine care products, including pads, tampons and liners, Depend® undergarments, underwear and guards, all available from Kimberly-Clark Corporation, Neenah, Wis.

It should be understood that the term “personal care product” as used herein refers a personal care article and a wrapper which is supplied to a consumer or user of the personal care articles as an individual product or as part of a package containing more than one personal care products.

As used herein, the term “nonwoven fabric or web” means a web having a structure of individual fibers or threads which are interlaid, but not in a regular or identifiable manner as in a knitted fabric. The term also includes individual filaments and strands, yarns or tows as well as foams and films that have been fibrillated, apertured, or otherwise treated to impart fabric-like properties. Nonwoven fabrics or webs have been formed from many processes such as for example, meltblowing processes, spunbonding processes, airlaying processes and bonded carded web processes. The basis weight of nonwoven fabrics is usually expressed in ounces of material per square yard (osy) or grams per square meter (gsm) and the fiber diameters useful are usually expressed in microns. (Note that to convert from osy to gsm, multiply osy by 33.91).

As used herein, the term “polymer” generally includes but is not limited to, homopolymers, copolymers, such as for example, block, graft, random and alternating copolymers, terpolymers, etc. and blends and modifications thereof. Furthermore, unless otherwise specifically limited, the term “polymer” shall include all possible geometrical configuration of the material. These configurations include, but are not limited to isotactic, syndiotactic and random symmetries.

As used herein, “body-facing surface” means the surface of the article or component which is intended to be disposed toward or placed adjacent to the body of the wearer during ordinary use, while the “outward surface” or “outward-facing surface” is on the opposite side, and is intended to be disposed to face away from the wearer's body during ordinary use. The outward surface may be arranged to face toward or placed adjacent to the wearer's undergarments when the absorbent article is worn.

As used herein, the term “sensory attribute” means a physical feature of the second panel or first panel of the wrapper component which may be perceived by a user. Physical features include, for example, color, texture, shape, a graphic, text, alpha-numeric characters, and/or a pattern, or other similar physical features.

As used herein, the term “sensory cue” is intended to mean information provided to a user of the products which distinguishes the second panel from the first panel. A sensory cue is provided to the user of the product by having different sensory attributes on the second panel and the first panel which allow a user to clear indicia as to the location of the free edge of the second panel so that the wrapper may be opened.

As used herein, “clear indicia” means that at a first encounter with the wrapper, a user can easily ascertain the location of the free edge of the second panel. Stated another way, the difference in the sensory attribute of the second panel and the sensory attribute of the first panel of the wrapper is such that the free end of the second panel is clearly apparent where the second panel and first panel are adjacent, which easily draws the user's attention to the free edge of the second panel.

As used herein, the phrase “how to open” is intended to mean that the package conveys a message, directly or indirectly, to the user of how the wrapper component is intended to be opened. For example, an envelope shape conveys to a user that there is a second panel and that the second panel may be opened by lifting the second panel upward.

As used herein, the term “connected” is intended to mean directly connected and indirectly connected. By directly connected, it is intended that the connected elements are in contact with one another or affixed to one another. By indirectly connected, it is intended that one or more intervening or intermediate elements are between the two elements which are secured or “connected” together. The intervening elements may be affixed.

### DETAILED DESCRIPTION OF THE INVENTION

Disposable absorbent articles such as, for example, feminine care and incontinent absorbent products, generally include a liquid pervious topsheet, a substantially liquid impervious backsheet, and an absorbent core positioned and held between the topsheet and the backsheet. The topsheet is generally operatively permeable to the liquids that are intended to be held or stored by the absorbent article, and the backsheet may be substantially impermeable or otherwise operatively impermeable to the liquids intended to be held or stored. Disposable absorbent articles may also include other optional components or layers, such as liquid wicking layers, liquid distribution layers, barrier layers, and the like, as well as combinations thereof, which may improve the fluid handling and storage properties of the disposable absorbent article. Generally, disposable absorbent articles and the components thereof provide a body-facing surface and a garment-facing surface. As an alternative, the substantially liquid impervious backsheet may be replaced with a liquid pervious backsheet, when the absorbent personal care product is used in conjunction with another liquid impervious layer or article, such as, for example liquid impervious pants.

In the present invention, the absorbent articles of the present invention are placed into a wrapper having an opening feature. The opening feature is highlighted to the user of the absorbent article by providing a sensory cue to the user. The sensory cue can be a visual cue, a tactile cue, an audible cue, an olfactory cue, a cue which invokes the sense of taste or a combination of one or more of these cues. From a practical standpoint, the visual or tactile cues are desirable.

To obtain a better understanding of the absorbent articles which may be present in the wrapper component of the present invention, attention is directed to FIG. 1. In FIG. 1, an exemplary absorbent product 10 is shown as including an outer cover 46 (otherwise referred to as a baffle or backsheet), an absorbent core 48, an optional tissue layer 6, an optional surge layer or option distribution layer 4 and a body side liner 44 (also referred to as the top sheet). The absorbent product 10 also has a first side 16 and a second side 18. The first and second sides 16, 18, respectively, are the longitudinal sides of the elongated absorbent product. The sides can be contoured, for example in a concave shape, or they can be linear. The sides can further include flaps (not shown) that extend laterally outward. Flaps are known in the art are shown in, for example U.S. Pat. No. 6,387,084 issued to VanGompel et al., which is hereby incorporated by reference in its entirety. In one embodiment (not shown), one or more elastic elements are disposed along the sides to form a gasket with the body of the user. Elastic sides are known in the art, as is shown in U.S. Pat. No. 6,315,765 issued to Datta et al., which is hereby incorporated by reference in its entirety. In one embodiment, the elastic elements are disposed between the body side liner and the outer cover.

The absorbent product 10 has a first body facing surface 20, which usually includes the outer surface of the body side liner 44, and a second garment facing side surface 22, which usually contains an outer portion of the outer cover 46. Applied to at least a portion of the second garment side surface 22 is a garment attachment adhesive. In various embodiments, the garment attachment adhesive is configured as a single band of adhesive or as two or more spaced apart strips. Alternatively, the garment attachment adhesive includes a swirl pattern of adhesive which encompasses a major portion of the second garment facing surface 22 of the absorbent article 10.

A release strip 28, also known as a releasable peel strip, is removably secured to the garment attachment adhesive and serves to prevent premature contamination of the adhesive before the absorbent article 10 is secured to the crotch portion of an undergarment. In various embodiments, the garment attachment adhesive is designed to be secured to the inner crotch portion of an undergarment so as to keep the absorbent product in register with the body of the user. The release strip 28 may extend beyond one or both of the ends 12, 14 of the outer cover, as shown in FIG. 1. As an alternative, the release strip may be shorter than the ends of the outer cover 12 and 14 (not shown). Generally, the only requirement for the length of the release strip 28 is that the release strip covers the garment adhesive often present on the outer cover 46.

The body side liner or topsheet 44, which is preferably liquid permeable, may be formed from one or more materials. The body side liner or topsheet 44 must be able to manage different body excretions depending on the type of product. In feminine care products, often the body side liner or body contacting layer must be able to handle menses and urine. In the present invention, the body side liner or topsheet 44 may include a layer constructed of any operative material, and may be a composite material. For example, the liner or body contacting layer can include a woven fabric, a nonwoven fabric, a polymer film, a film-nonwoven fabric laminate or the like, as well as combinations thereof. Examples of a nonwoven fabric useable in the body side liner or topsheet 44 include, for example, an airlaid nonwoven web, spunbond nonwoven web, meltblown nonwoven web, a bonded-carded-web, hydroentangled nonwoven webs, spunlace webs or the like, as well as combinations thereof. Other examples of suitable materials for constructing the body side liner or topsheet 44 can include rayon, bonded carded webs of polyester, polypropylene, polyethylene, nylon, or other heat-bondable fibers finely perforated film webs, net-like materials, and the like, as well as combinations thereof. These webs can be prepared from polymeric materials such as, for example, polyolefins, such as polypropylene and polyethylene and copolymers thereof, polyesters in general including aliphatic esters such as polylactic acid, nylon or any other heat bondable materials.

Other examples of suitable materials for the body side liner or topsheet 44 are composite materials of a polymer and a nonwoven fabric material. The composite materials are typically in the form of integral sheets generally formed by the extrusion of a polymer onto a nonwoven web, such as a spunbond material. In a desired arrangement, the body side liner or body contacting layer 44 can be configured to be operatively liquid-permeable with regard to the liquids that the article is intended to absorb or otherwise handle. The operative liquid-permeability may, for example, be provided by a plurality of pores, perforations, apertures or other openings, as well as combinations thereof, which are present or formed in the liner or body contacting layer. The apertures or other openings can help increase the rate at which bodily liquids can move through the thickness of the body side liner or body contacting layer and penetrate into the other components of the article (e.g., into the absorbent core 48). The selected arrangement of liquid-permeability is desirably present at least on an operative portion of the body side liner or topsheet 44 that is appointed for placement on the body-side of the article. The body side liner or topsheet 44 can provide comfort and conformability, and can function to direct bodily exudates away from the body and toward the absorbent core 48. The body side liner or topsheet 44 can be configured to retain little or no liquid in its structure, and can be configured to provide a relatively comfortable and non-irritating surface next to the body-tissue of a wearer.

The baffle or backsheet 46 may include a layer constructed of any operative material, and may or may not have a selected level of liquid-permeability or liquid-impermeability, as desired. In a particular configuration, the baffle or backsheet 46 may be configured to provide an operatively liquid-impermeable baffle structure. The baffle or backsheet 46 may, for example, include a polymeric film, a woven fabric, a nonwoven fabric or the like, as well as combinations or composites thereof. For example, the baffle may include a polymer film laminated to a woven or nonwoven fabric. In a particular feature, the polymer film can be composed of polyethylene, polypropylene, polyester or the like, as well as combinations thereof. Additionally, the polymer film may be micro-embossed, have a printed design, have a printed message to the consumer, and/or may be at least partially colored. Suitably, the baffle or backsheet 46 can operatively permit a sufficient passage of air and moisture vapor out of the article, particularly out of an absorbent (e.g. storage or absorbent core 48) while blocking the passage of bodily liquids. An example of a suitable baffle material can include a breathable, microporous film, such as those described in, for example, U.S. Pat. No. 6,045,900 to McCormack et al.

Bicomponent films or other multi-component films can also be used, as well as woven and/or nonwoven fabrics which have been treated to render them operatively liquid-impermeable. Other suitable baffle materials may be used, including closed cell polyolefin foams. For example, a closed cell polyethylene foam may be employed.

The liquid permeable body side liner 44 and the liquid-impermeable baffle 46 may be peripherally sealed together to enclose the absorbent core 48 to form the absorbent article 10. Alternatively, the body side liner or topsheet 44 can be wrapped around both the absorbent 48 and the baffle or backsheet 46 to form a wrapped pad. The body side liner 44 and baffle 46, and other components of the absorbent product, can be joined for example with adhesive bonds, ultrasonic bonds, thermal bonds, pinning, stitching or any other attachment techniques known in the art, as well as combinations thereof.

The absorbent core 48 is designed to absorb body exudates, including menstrual fluid, blood, urine, and other body fluids. The absorbent core 48 may contain one or more layers of absorbent material. The layers can contain similar materials or different materials. Suitable materials for the absorbent core 48 include, for example, cellulose, wood pulp fluff, rayon, cotton, and meltblown polymers such as polyester, polypropylene or coform. Coform is a meltblown air-formed combination of meltblown polymers, such as polypropylene, and absorbent staple fibers, such as cellulose. A preferred material is wood pulp fluff for its low cost, relative ease of formation and good absorbent properties.

The absorbent core 48 can also be formed from a composite comprised of a hydrophilic material which may be formed from various natural or synthetic fibers, wood pulp fibers, regenerated cellulose or cotton fibers, or a blend of pulp and other fibers. A desired material is an airlaid material.

In one embodiment, the absorbent core 48 also includes a superabsorbent material, in addition to or in place of the hydrophilic material, which increases the ability of the absorbent core to absorb a large amount of fluid in relation to its own weight. Generally stated, the superabsorbent material can be a water-swellable, generally water-insoluble, hydrogel-forming polymeric absorbent material, which is capable of absorbing at least about 15, suitably about 30, and possibly about 60 times or more its weight in physiological saline (e.g. saline with 0.9 wt % NaCl). The superabsorbent materials can be inserted as particles or in sheet form. The superabsorbent material may be biodegradable or bipolar. The hydrogel-forming polymeric absorbent material may be formed from organic hydrogel-forming polymeric material, which may include natural material such as agar, pectin, and guar gum; modified natural materials such as carboxymethyl cellulose, carboxyethyl cellulose, and hydroxypropyl cellulose; and synthetic hydrogel-forming polymers. Synthetic hydrogel-forming polymers include, for example, alkali metal salts of polyacrylic acid, polyacrylamides, polyvinyl alcohol, ethylene maleic anhydride copolymers, polyvinyl ethers, polyvinyl morpholinone, polymers and copolymers of vinyl sulfonic acid, polyacrylates, polyacrylamides, polyvinyl pyridine, and the like. Other suitable hydrogel-forming polymers include hydrolyzed acrylonitrile grafted starch, acrylic acid grafted starch, and isobutylene maleic anhydride copolymers and mixtures thereof. The hydrogel-forming polymers may be lightly crosslinked to render the material substantially water insoluble. Crosslinking may, for example, be by irradiation or covalent, ionic, Van der Waals, or hydrogen bonding. Hydroxyfunctional polymers have been found to be good superabsorbents for sanitary napkins. Such superabsorbents are commercially available from Dow Chemical, Hoechst-Celanese, and Stockhausen, Incorporated, among others, and are a partially neutralized salt of cross-linked copolymer of polyacrylic acid and polyvinyl alcohol having an absorbency under load value above 25 grams of absorbed liquid per gram of absorbent material (g/g). Other types of superabsorbent materials known to those skilled in the art can also be used.

Additional layers or substrates, including for example, the liquid acquisition and distribution layer 4, also referred to as a surge or transfer layer, and an optional tissue layer 6 are also incorporated into the absorbent product, for example, between the body side liner or topsheet 44 and the absorbent core 48. The distribution layer 4 may be shorter than the absorbent core 48 or have the same length as the absorbent core 48. The distribution layer serves to temporarily hold an insulting fluid to allow the absorbent core sufficient time to absorb the fluid, especially when a superabsorbent material is present. In one embodiment, the absorbent core, distribution layer and other components, such as tissue layers, are free floating (unattached) between the outer cover and the liner, which are secured along only the peripheral edges thereof. Alternatively, the absorbent core, transfer layer and other components are attached to one or both of the outer cover and liner and/or to each other.

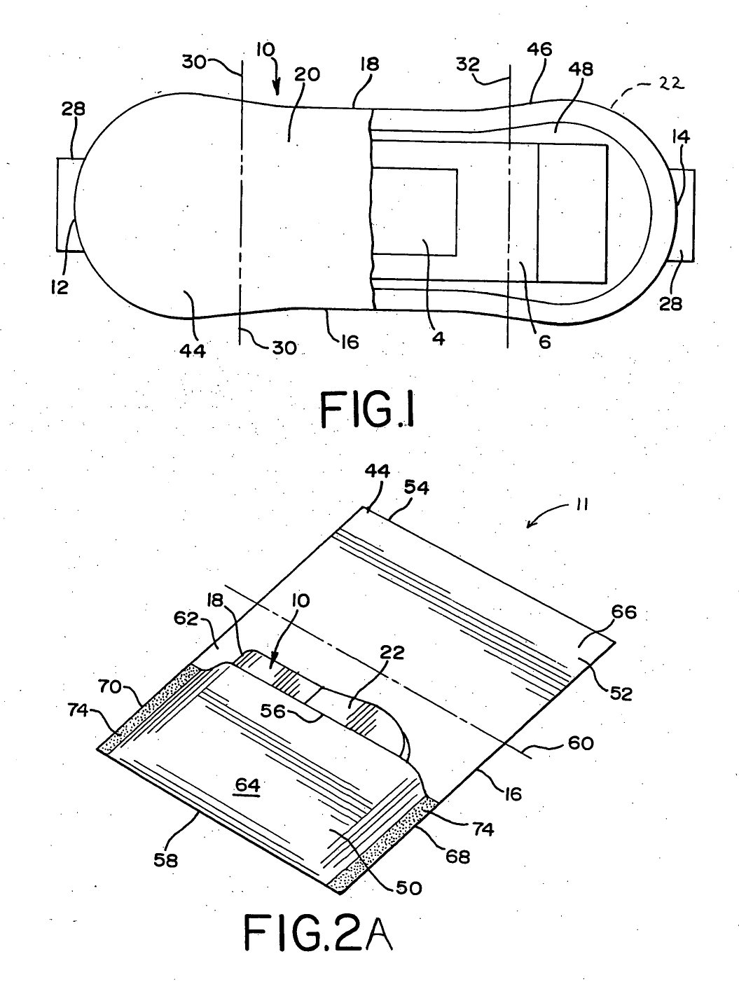

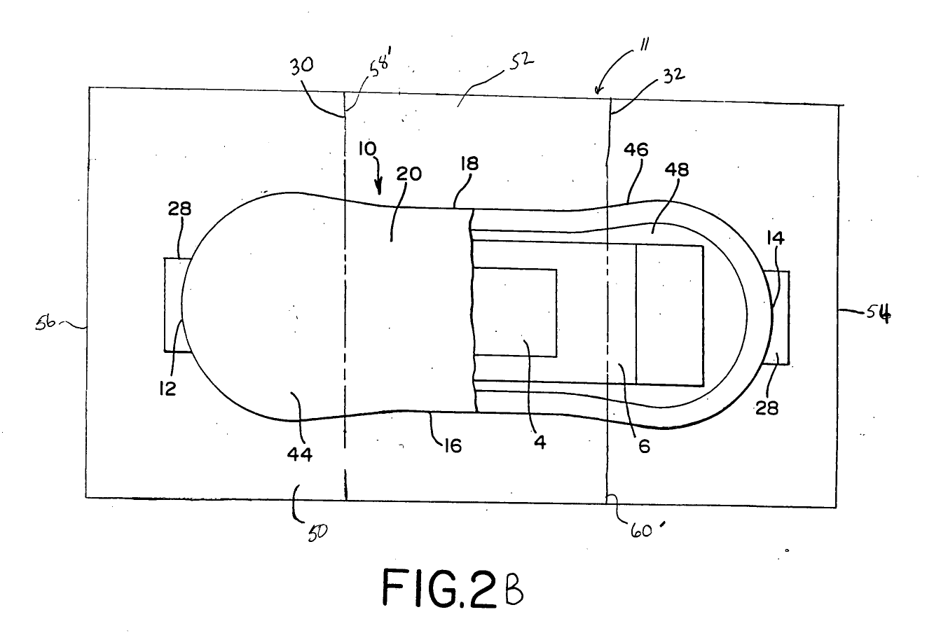

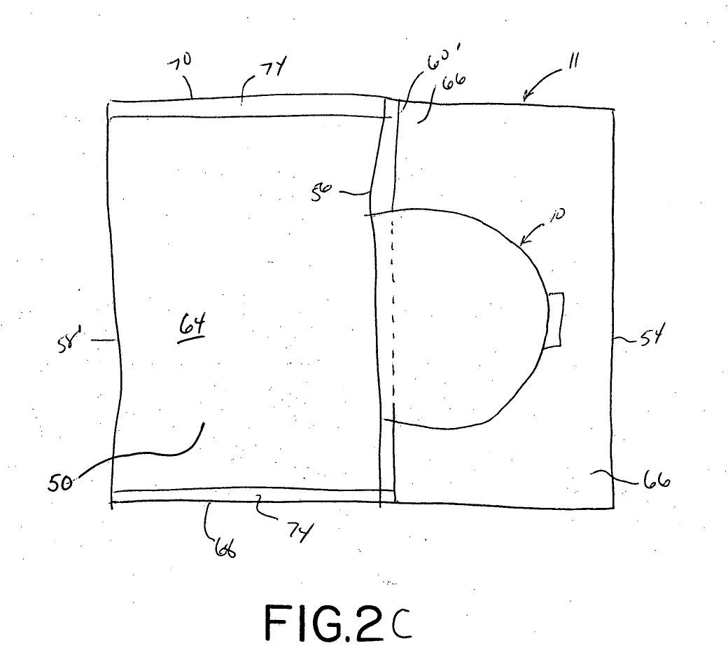

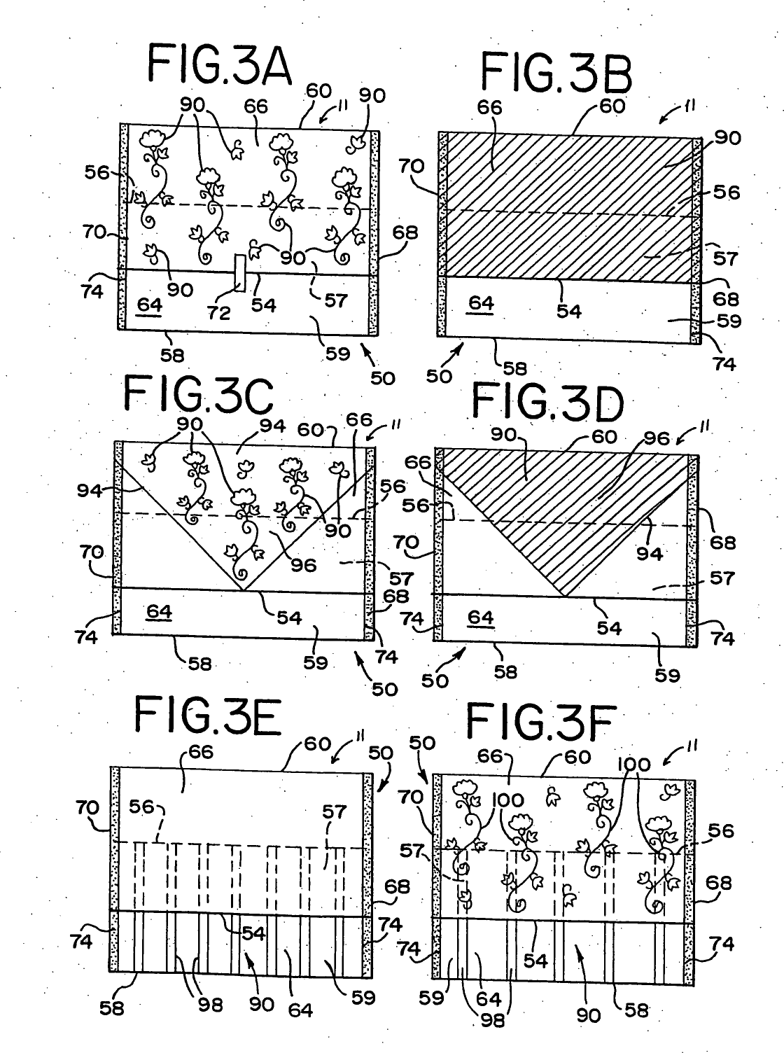

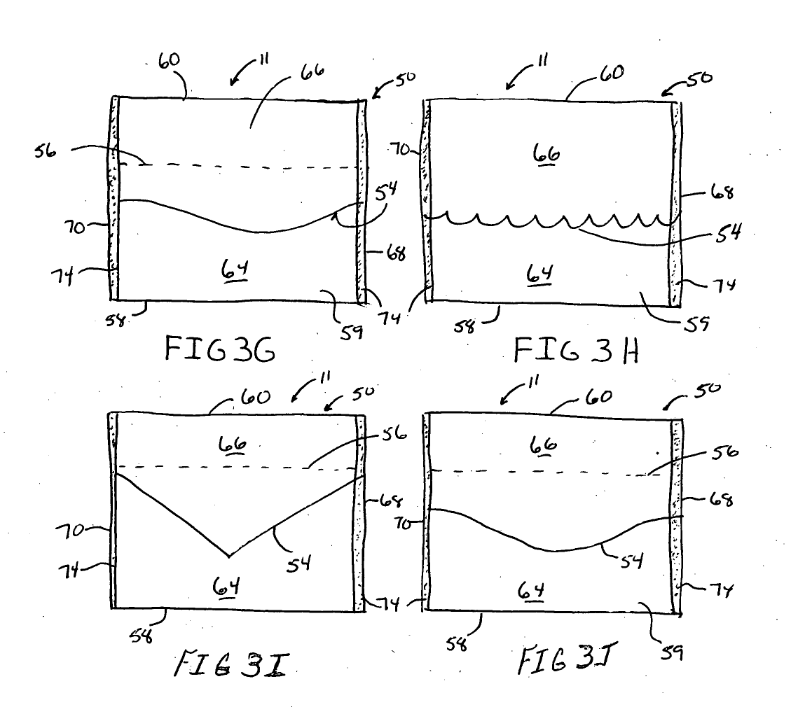

Although the wrapper component may have different configurations or can be prepared in other ways without departing from the scope of the present invention, referring to FIGS. 2A, and 3, a typical wrapper component 11 has a pouch 50 formed from a strip or web 52 of material having a first and second ends having free edge 56, 54, respectively. It should be understood that the term “free edge” refers to an edge that is unattached after the wrapper component is opened, regardless of whether the free edge is attached when the wrapper component is closed. Each of the first and second ends is folded along fold lines 58′, and 60′ that define the top 60 and bottom edge 58 of the pouch 50, respectively. The folded pouch has a back panel 62, a first panel 64 and a second panel 66. The first panel 64 and back panel 62 may be secured along side edges 68, 70 thereof to form a pocket shape to receive the absorbent product. In one embodiment, the pocket and pouch may be shaped and dimensioned to receive a single product component, which is individually wrapped in the pouch. Alternatively, the pocket and pouch may be configured to hold more that one absorbent article. The second panel 66 is folded over the first panel 64 such that the free edge 54 of the second panel overlies the first panel 64. The first panel 64 has a covered or overlap portion 57 extending between the free edge 54 (exterior) and the free edge 56 (interior), which covered or overlapped portion 57 underlies the second panel 66. Generally, the second panel may be a flap, but it is not required for the present invention that the second panel be flap. A flap is a piece of the wrapper component which may be manipulated by a user by grasping the end of the flap and pulling the flap upward, towards the top 60.

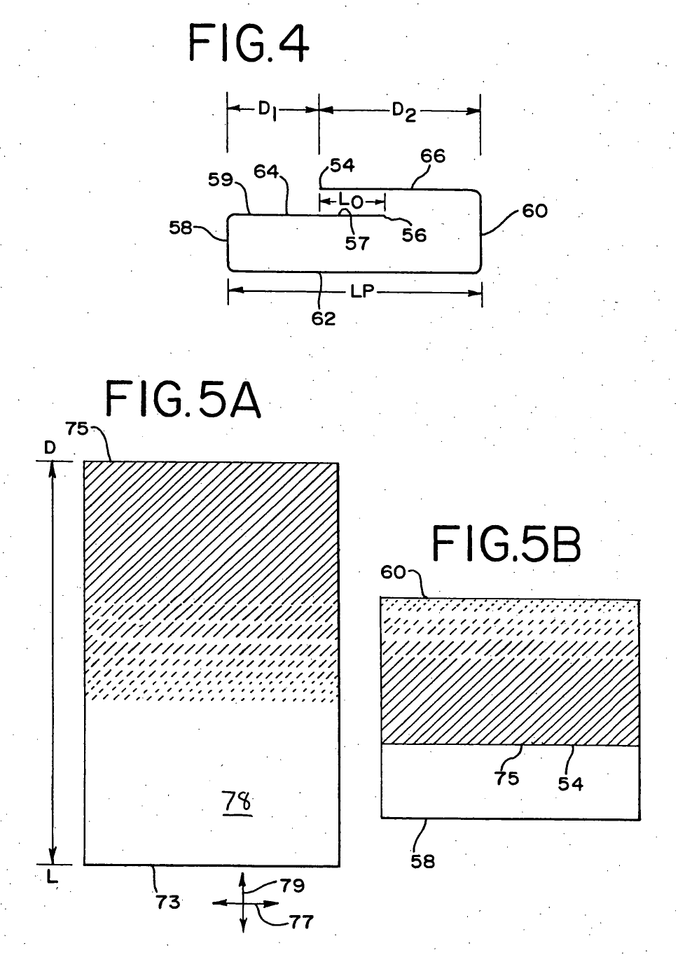

When the second panel overlaps the first panel, the overlap distance (Lo) is less than or equal to about 95% of the overall wrapper component length (Lp) in a closed configuration (shown in FIG. 4), more desirable less than or equal to about 35% of the wrapper component length, and more desirably less than or equal to about 20% of the wrapper component length. In various embodiments, the free edge 54 is positioned at a distance (D1, D2) from either edge 58, or 60 that is greater than or equal to about 10% of the overall length (Lp) of the wrapper component (in a closed configuration), more desirably greater than or equal to about 30%, and more desirably about 50% of the wrapper component length. The first panel further includes an uncovered second portion 59 extending between the free edge 54 and the bottom edge 58. Of course, it should be understood that the length and width of the product and wrapper components can vary according to the type of product and the size of the product. In one example, in the case of feminine napkins or incontinence pads, the portion 57 has a length (Lo) of about 0 to about 50 mm, generally between about 2-22 mm and typically about 4-10 mm, between the edges 54, 56.

A pair of side seals 74 secures the first panel 64 to the back panel 62. The side seals are desirably formed after the first panel is folded over the back panel and the second panel is folded over the back panel and the first panel. Although, it is possible that the first panel could first be sealed to the back panel, and the second panel then sealed to one or both of the back panel and first panel. In an alternative configuration, the second panel is not sealed at the side edges of the first panel and back panel. This embodiment is described in more detail below. The sides may be sealed by any method known to those skilled in the art. Exemplary sealing methods include, for example, adhesive sealing, bonding by the application of heat and pressure, ultrasonic bonding or any other art known bonding methods. In one embodiment of the present invention, the side seals 74 may be frangible, meaning they can be easily broken such that the second panel 66 can be separated from the first panel 64 and back panel 62, and such that the first panel 64 can be easily separated from the back panel 62, wherein the product component 10 is exposed for removal from the pouch by the user.

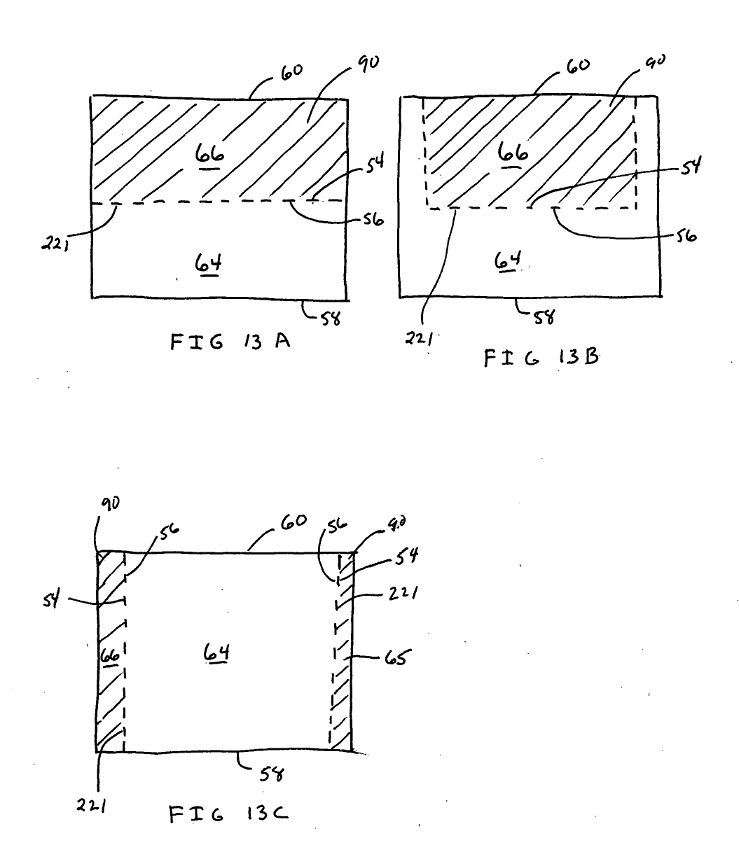

Accordingly, one or more free edges may be formed along a perforation line, which is shown in FIG. 13A-13C, or may be adhered to an underlying layer, with the edge defined by the perforation line being a “free edge” after the perforation line is broken. The free edge can be a single layer cut or formed edge, or can include a double-layer folded edge, or can include an edge formed by a plurality of layers.

The wrapper material can be formed from materials, such as, but not limited to, a non-woven material, films, paper, laminates, and/or cloth (including woven) materials, and combinations thereof. For example, the pouch can be made as disclosed in U.S. Pat. No. 6,716,203, to Sorebo et al., the entire disclosure of which is hereby incorporated herein by reference. Suitable laminates useable in the present invention include, spunbond-spunbond laminate (SS), spunbond-meltblown-spunbond laminates (SMS), spunbond-film laminates (SF), and film-film laminates. In one embodiment, the pouch is made of a film/spunbond laminate material available from Kimberly-Clark Corp, and known as HBSTL (“highly breathable stretch thermal laminate”), and which material is further disclosed in U.S. Pat. No. 6,276,032, the entire disclosure of which is hereby incorporated herein by reference.

In one alternative embodiment, the second panel 66 is releasably secured to the first panel 64. For example, a fastening element 72, shown as a tab in FIG. 3A, is secured across the free edge 54 of the second panel 66 to secure the second panel 66 to the first panel 64. The fastening element can be releasably secured to both of the second panel and first panel, or it can be fixedly secured to one of the second panel and first panel and releasably secured to the other. Other possible configuration include that the fastening element is fixedly secured to both panels and one or both of the panels is provided with an area of weakness, such as a perforated area, which allows a portion of one or both of the panels to be removed or damaged when the wrapper is opened. The fastening element can be formed as adhesive tape, a snap, a button, a mechanical fastener (e.g., hook and loop), a tie, or as any other device known by those skilled in the art. The fastening element can have various alternative shapes, including but not limited to a square, rectangle, triangle, circle, oval, obround, oblong or diamond shape, or any other irregular shape or pattern. In an alternative embodiment, the fastening element is formed on the inside of the second panel such that it engages the first panel as the second panel is folded thereover and is not visible to the user. For example, the fastening of the second panel 66 to the first panel 64 may be accomplished by the use of an adhesive applied to the side of the second panel 66 which contacts the first panel 64, to the side of the first panel 64 which contacts the second panel 66, or both. This adhesive may be applied as a ribbon, dot, a swirl pattern or any other pattern which effectively adheres the second panel 66 to the first panel 64. In another alternative way to fasten the second panel 66 to the first panel 64, the second panel 66 is simply sealed to the first panel 64 with a heat seal or other weld, with the weld defining the fastening element. In another embodiment, the second panel 66 is not sealed or otherwise attached to the first panel 64, but rather is simply folded thereover. Alternatively, the sides of the second panel are sealed to the back panel and to the first panel, with the side seals being breakable in response to a user grasping and lifting the second panel. It is within the scope of the present invention that the sensory cue could be located on the fastening element.

In some embodiments, the second panel 66 is refastenably secured to the first panel 64, while in others, the second panel 66 is not intended to be secured to the first panel 64 once the wrapper component is opened. For example, in one embodiment, the free edge 54 is defined by a perforation line, with the second panel not being refastenable after the perforation is broken.

Referring to FIGS. 1 and 2A, the absorbent product 10 is shown in a folded configuration. For example, the absorbent product can be folded along a pair of fold lines 30, 32 to form a tri-fold configuration. In other embodiments, the absorbent product can be bi-folded, flat or rolled. The absorbent product is then inserted into an individual wrapper component 11, otherwise referred to as a wrapper or pouch. Alternatively, a plurality (meaning two or more) of products 10 can be disposed in a wrapper component 11. One product/wrapper configuration is shown in U.S. Pat. No. 6,601,706 to McManus et al., which is hereby incorporated by reference. Other wrapper/absorbent product configurations are described in, for example U.S. Patent Application Publication 2002/0079246 to Ling et al., which is hereby incorporated by reference. The absorbent products can be oriented in various ways within the individual wrapper component, for example, with the fold lines 30, 32 running parallel or perpendicular to the sides of the wrapper component 68, 70.

Another wrapper component embodiment of the present invention is shown in FIGS. 2B and 2C. In this embodiment, the wrapper component 11 and the absorbent article 10 are folded together. The wrapper component 11 is a strip or web 52 of material having a first and second ends having free edge 54, 56, respectively. First, the wrapper component and the absorbent article are folded together along line 30 such that the body side liner or topsheet 44 of the absorbent article comes in contact with itself. In addition, each of sides 68 and 70 come into contact with itself and may be sealed together to form two side seals 74, as is shown in FIG. 2C, or can remain unsealed at this stage. Generally, the wrapper component sides are bonded in the side regions 68 and 70. The sides may be sealed by any method known to those skilled in the art including adhesive sealing, bonding by the application of heat and pressure, ultrasonic bonding or any other art known bonding methods. When folded and sealed as shown in FIG. 2C, the wrapper component has a pouch with a back panel 62, a first panel 64 and a second panel 66. The second panel 66 is then folded over the first panel 64 along line 32 such that the free edge 54 of the second panel overlies the first panel 64. In this configuration, the portion of the top sheet of the absorbent article will be in contact with the first panel 64 of the wrapper component 11. As with the above described configuration, the first panel 64 has a covered or overlapped portion 57, shown in FIG. 4, extending between the free edge 54 (exterior) and the free edge 56 (interior), wherein the covered or overlapped portion 57 underlies the second panel. It may be advantageous from a materials standpoint to fold the wrapper component 11 and the absorbent article together, since the release strip 28 may be unnecessary since the wrapper component 11 could also be used as the release strip. As with the previous described configurations, the wrapper component may be designed such that there is no overlap between the free edge 54 and the first panel 64, without departing from the scope of the present invention. For example, the free edges 54, 56 may abut each as shown in FIG. 13A other or may be spread apart by a small distance (not shown). As such, in this alternative, the second panel is defined merely as another panel.

In other wrapper configurations shown in FIGS. 13A, 13B and 13C, the free edge 54 of the second panel 66 abuts the free edge 56 of the first panel. A perforation line 221 or other line of weakness separate the first panel 64 and second panel 66 from one another and the free edges 54 and 56 are formed when the perforation 221 or line of weakness is broken. In addition, as shown in FIG. 13C, the first panel 64 of the wrapper component may have more than one free edge 54 and there may be more than two panels present on the wrapper component. A third panel 65 is provided. Further, as is shown in FIG. 13B, the second panel 66 does not need to extend the entire width or length of the wrapper component. The second panel 64, or maybe a portion of the width of the wrapper component. The wrapper configurations shown in FIGS. 13A, 13B and 13C are described in further detail in U.S. Pat. No. 6,036,679 to Balzar et al, which is hereby incorporated by reference.

In yet another possible wrapper configuration, the wrapper component is formed as a portion of the product component. For example, an outer cover, such as the baffle material, can form a wrapper component, as shown for example in U.S. Pat. No. 5,993,430 to Gossens, which is hereby incorporated herein by reference. It should be understood that in these embodiments, the product component is considered to be disposed in the wrapper component when the wrapper element is sealed or otherwise closed. In other embodiments, the product component is rolled, with a flap or front panel portion of a wrapper component overlying a panel portion of the wrapper component, regardless of whether the wrapper component is integral with the product component or separate therefrom.

The present invention provides a sensory cue to the user of the absorbent article, indicating to the user where the free edge of the second panel of the wrapper component is located. The sensory cue can be a visual cue, a tactile cue, an audible cue, an olfactory cue, a cue which invokes the sense of taste or a combination of one or more of these cues. From a practical standpoint, visual or tactile cues are desirable. This sensory cue aids the user of the absorbent article to locate the place on the wrapper component in which the wrapper component may be opened to access the absorbent article contained within the wrapper.

From a practical standpoint, the visual or tactile cues are desirable. However, this does not mean that other sensory cues are intended to be excluded from the present invention. For example, audible cues, olfactory cues and cues which invoke taste can be very useful for users of the products who are severely visually impaired to the point that they are blind or essentially blind. In the case of taste, a flavoring could be placed on the edge of the flap or second panel near the opening to allow a user to use the sense of taste to find the opening for the pouch. Likewise, a “scratch and sniff” type coating could be placed near the opening of the pouch to allow a user to use the sense of smell to locate the opening. In a similar manner, the audible means could be used in the present invention by changing the type of material or thickness of the material used for the flap or second panel so that when touched or handled, the sound generated by the flap or second panel is different from the remainder of the pouch material.

In the present invention, the sensory cue is provided by a contrast between the first panel and the second panel of the wrapper. This may be accomplished by providing the second panel with a first set of sensory attributes and the first panel with a second set of sensory attributes. At least one attribute of the first set of sensory attributes or the second set of sensory attributes is different from the other sensory attributes of the other set of sensory attributes. In the present invention, the difference in sensory attributes on the second panel and first panel must provide clear indicia as to where the free edge is located at a first encounter with the wrapper component That is, the user should not have to think about the location of the free edge based on the sensory attributes on the flap or wrapper. In this regard, attention is directed to FIG. 10A and FIG. 10B which show prior art wrapper configurations. Each wrapper 11′ has a flap 66′ and a front panel 64′. A sensory attribute 90′ is applied the bottom portion of the flap 66′ along the free edge 54″. A user first looking at this configuration does not initially glean from the configuration the location of the free edge 54″. The free edge could be located above the sensory attribute 90′ or below the sensory attribute 90′. In this case, one is not provided with clear indicia as to the location of the free edge, hence these configurations are outside the present invention. Further, FIG. 10B further confuses the user as to whether the opening is below the sensory attribute or above. One could construe the downward arrow 90″ to indicate that the flap 66′ is to be opened downward.

Sensory attributes can take many forms and can include, for example, one or more colors, textures, shapes, graphics, text, alpha-numeric characters, and/or patterns, including indicia formed by dying, printing and/or embossing, or by otherwise altering the relative texture of the second panel relative to the first panel. Other examples include second panel and first panel material type, second panel and first panel material color, patterns embossed or printed onto the second panel or first panel, shape of the free edge, or any other attribute which may allow a user to ascertain a difference between the second panel and the first panel and enable the user to recognize the free edge of the second panel by the contrast created by the difference in at least one attribute.

To gain a better understanding of the sensory cue provided by the present invention, attention is directed to FIGS. 3A-3J. In each of FIGS. 3A-3J, at least one of the second panel 66 and the first panel 64 includes a sensory attribute 90, shown as a visual or tactile attribute. The sensory attribute 90 in FIG. 3A is present on the second panel 66 but not on the first panel 64. The sensory attribute 90 desirably terminates at the free edge 54 and provides indicia as to the location of the free edge. With the sensory attribute only on the second panel, a sensory cue is provided to a user, directing the user's attention to the free edge 54, where the sensory attribute on the second panel 66 ends and the first panel 64 begins. The terms “directed at” and “terminates” do not require that the sensory cue created or the sensory attribute necessarily contact or run all the entire distance of the free edge 54, as shown for example in FIGS. 3A and 3C, wherein a pattern 92 is applied over portions of the second panel 66, but with a lowermost portion thereof being slightly spaced from the edge 54. As is stated above, the sensory attributes 90 can take many forms, and can include one or more colors, textures, shapes, graphics, text, alpha-numeric characters, and/or patterns, including indicia formed by dying, printing and/or embossing, or by otherwise altering the relative texture of the second panel relative to the first panel. The sensory attributes can further be reflective and/or glow in the dark such that the user can access the free edge in poorly lit conditions.

For example, in the embodiment of FIG. 3A, the sensory attribute 90 is configured as a decorative pattern applied to the entirety of the second panel 66. In one suitable embodiment, the pattern is formed by repeating pictorials. Of course, it should be understood that the pattern can be any combination of repeating pictorials, lines, shapes, characters, etc. In the embodiment of FIGS. 3B, 13A, 13B and 13C, the sensory attribute 90 is configured as a color applied to the second panel 66 that is different than the color of the first panel 64, meaning the colors have a different hue. One or more colors may also be different by virtue of having a different luminosity and/or saturation/vividness. Saturation/vividness is the intensity of the color from pale to dark. The elements may also have a different gloss/finish, from a matte finish, which tends to diffuse or scatter light, to a gloss finish, which is specular.

Referring to FIG. 8, hue is measured by the angular position around the circle 110. Two colors are considered different if they have first and second hues that are more than ±0.50 degrees from each other on the circle 110, alternatively ±5 degrees, alternatively ±30 degrees, alternatively ±90 degrees, alternatively ±150 degrees and alternatively ±175 degrees. Value (luminosity) is measured along the Z-axis 112 between white and black. Colors are considered different if they have a value difference of at least 1% of maximum (Polaroid white reference standard). Desirably, the value difference should be at least 20% of the maximum and more desirably at least 33% of the maximum. A value of one equates to white, while a value of 0 equates to black. Saturation/vividness is measured along the length of the radius (r). Colors are considered different if they have a saturation difference of at least 2.5% of maximum. Desirably, the saturation difference should be at least 7% and more desirably at least 33% of the maximum.

The hue, luminosity and saturation/vividness are measured as follows using the following equipment calibrated in the following way.

Equipment

Quantitative colorimetric measurements are typically made using a colorimeter or spectrophotometer. However, these instruments typically have large apertures (˜1 cm) requiring a large color block for meaningful determination, making them unsuitable for color determination of graphics that may be composed of narrow lines or points whose width is much less than the instrument aperture. Therefore, a Zeiss KS400 Image Analysis system was used for feature identification and calorimetric measurement.

The Zeiss KS400 used a Zeiss AxioCam color CCD camera (1300×1030 pixels, 3 channel color, 8 bit per channel) equipped with a 20 mm AF-Nikkor lens (f/2.8). The camera was mounted vertically facing down onto a sample stage and had an effective field of view was 97×80 mm. Incident sample stage illumination was by four incandescent floodlamps (Sylvania) on a double Variac (70%; 90%), resulting in an illuminance of approximately 11,000 lux. The lamps were above the left and right edges of the sample stage directed towards the field of view at approximately 45 degrees.

Calibration

The camera black reference was with the lens cap on. The camera white reference was a Polaroid 803 positive with 15 ms exposure. To account for the warm color illumination bias of the floodlamps, the red, green, and blue (RGB) values were offset using the white selection tool in the KS400 software, resulting in corrected RGB values that yielded a white image.

Sample Setup and Image Acquisition

Samples are placed on the stage (normal viewing angle) and under ¼″ plate glass to minimize topographical effects. Images of the color-bearing graphical portion are acquired at 15 ms exposure.

Image Analysis

Image analysis is performed in Matlab (v.6.5.1, release 13; Mathworks, Inc) with the Image Processing Toolbox (v4.0). RGB images were converted to hue, saturation, and value (HSV) space using Matlab's hsv2rgb.m command. Choosing a saturation lower limit of 0.05 (0-1 scale) resulted in practical detection of all the colored/inked portions of the graphic. The hue, saturation, and value (i.e. luminosity) densiometric distributions were calculated for the detected regions in each image.

Referring to FIG. 3C, the sensory attribute 90 includes a pattern formed over a portion of the second panel 66. In FIG. 3D, the sensory attribute 90 is configured as a color applied to portion of the second panel 66. In addition, an arrow shaped graphic 94 is pointed at the edge 54. The arrow shaped graphic 94 defines a sensory attribute region 96, which can include a pattern, as shown in FIG. 3C, or be of a different color, as shown in FIG. 3D, than the remainder of the second panel 66. This further directs the user's attention to the edge 54. It is also pointed out that with the arrow shaped graphic, the second panel resembles and letter-type envelope, which tends to direct the user which direction the second panel should be opened. That is, unsealed and sealed envelopes are typically opened from the point to the top. Therefore, the sensory cue could also provide a means for directing the user to which way the second panel should be opened, in relationship to the first panel.

Referring to FIG. 3E, the first panel is configured with a pattern 98, shown as alternating and spaced apart thin and thick vertical lines, on the first panel 64 of the pouch, that underlies the second panel 66. In this embodiment, the second panel 66 with its plain configuration contrasted with the pattern 98 of the front portion, thereby providing a sensory cue to the user as to the location of the edge 54 of the second panel 66. The second panel 66 can be configured with any color, including white. Desirably, the color of the second panel is selected such that it will aid in the contrast of the second panel 66 from the first panel 64 of the pouch. In another alternative configuration, referring to FIG. 3F, the second panel 66 is configured with a first sensory attribute in the form of a pattern 100, and the first panel is configured with a second sensory attribute in the form of a pattern 98 that is different from the first pattern 100, thereby providing a sensory cue as to the location of the free edge 54 of the second panel 66, which is clearly located where the first sensory attribute and the second sensory attribute intersect.

It is pointed out that the patterns shown in FIGS. 3A, 3C, 3E and 3F can be printed onto the wrapper component material or can be embossed onto the wrapper component material. When embossed, both visual and tactile sensory cue will be provided. Tactile sensory cues can also aid the user of the product to locate the wrapper component with the absorbent article when stored in a location containing many objects, such as a purse, diaper bag or brief case. Further, using embossing instead of printing may also aid the user in opening the wrapper component since the embossing may allow the user to improve their grip on the second panel, as compared to an unembossed second panel. Embossing has other advantages over printing, including for example, lower material cost since inks or dyes would not be necessary impart the sensory cue. Since embossing provides both a visual and a tactile sensory cue, it allows all users of the absorbent article, including those with vision impairment, to locate the second panel of the wrapper component. Methods of embossing material are well known in the art. For example, the wrapper material may be passed between a heated or unheated anvil roll and heated or unheated embossing roll, which is engraved with a relief embossing pattern. Other techniques known to those skilled in the art, including using ultrasonic embossing and the like may also be used to obtain the desired embossing effect. The method in which the wrapper component is embossed is not critical to the present invention. Generally, it is desired, but not required, that the embossing pattern used contain objects which impart a feminine flair to the wrapper component, such as floral designs, when the absorbent articles are designed for use in feminine personal care articles.

Embossing can be varied on the wrapper component. For example, the second panel 66 may be embossed as shown in FIG. 3A or the first panel of the pouch 64 may be embossed with the pattern. In either event, the embossing pattern directs the user's attention to the edge 54, since the pattern terminates at the edge 54. The embossing pattern can be registered or non-registered and can be a high density pattern or a low-density pattern.

In yet another possible configuration for the sensory cue, the sensory attribute could include the edge 54 of the second panel 66 having a distinct shape compared to the remainder of the wrapper component. For example, the wrapper components generally have linear top 60, bottom 58 and side 70 and 68 edges, as is generally shown in FIGS. 3A-F. As a result of making the edge 54 of the second panel 66 non-linear, the edge provides a sensory cue as to the location of the opening of the wrapper. FIGS. 3G-3J show some possible nonlinear edges usable in the present invention. The sensory cue provided by the non-linear edge 54 is both visual and tactile. FIG. 3G shows the edge 54 as having a wave pattern. In FIG. 3H, the edge 54 is shown as having a scalloped shape. FIGS. 3I and 3J each show the second panel 66 as having a V-shape edge 54. Other non-linear shapes may be used without departing from the scope of the present invention. Another possible advantage of having a non-liner edge 54 on the second panel 66 of the wrapper is that the second panel can be easier to grasp for those users who have impaired hand strength, such as may be caused by arthritis and the like.

Of the above described shapes, the V-shape can be advantageous since the shape resembles an envelope. Most potential users of the absorbent products are familiar with an envelope shape and how to open an envelope. Therefore, by virtue of the V-shape, the user is directed how to open the wrapper component.

Another way to obtain a sensory cue which directs a user to the edge of the second panel of the wrapper component is to use a two-sided material as the pouch component. The term “two-sided”, as used herein, is intended to mean that the material has different attributes on each side of the material. The different attributes may be a different color, different surface texture, different materials on each side and the like. Generally, the wrapper component material which is two-sided is generally a laminated material such as, for example, a spunbond-film laminate (SF), a spunbond-meltblown-spunbond laminate (SMS), spunbond-spunbond laminate, a film-film laminate and the like.

By providing a material which is two-sided, the second panel and/or the first panel can easily be provided with a sensory cue directed towards the edge of the second panel. If each side of the material has a different color, when embossed, the colors may blend together, due to the melting of the materials of each side when embossed using heat, to give a third color which is different from the first two colors. For example, if one side of the material is white and the other side is blue, in the areas where the material is embossed, a second blue color may result which will be distinguishable from the first blue color. As a further example, if one side is yellow and the other side is blue, when embossed using heat and pressure, a green color may result. The embossing may take place on the second panel or the first panel of the pouch to provide the sensory cue to the user as to the location of the second panel opening. In addition, the embossing and color change will provide two separate sensory cues as to the location of the free edge of the second panel.

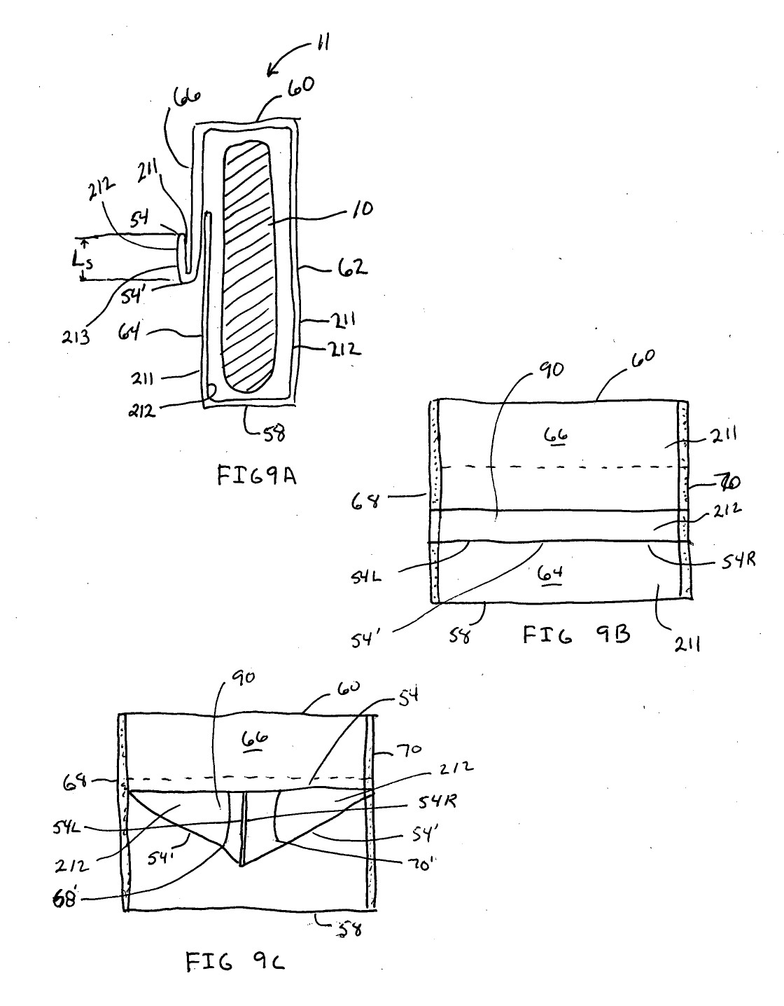

In another embodiment of the present invention, when each side of the wrapper material has different attributes, the edge of the second panel may be turned upward, so as to expose the side of the wrapper material which is adjacent the absorbent article inside the wrapper component. To gain a better understanding of some of the advantages of using a two-sided wrapper component material in the present invention, attention is directed to FIGS. 9A and 9B. As can be seen in FIG. 9A, which exaggerates the packing component to clearly show the structure, the wrapper component 11 has a first side 211 and a second side 212, with the first side 211 forming the bulk of the outside surface of the wrapper component 11, which the second side 212 forms the bulk of the inside surface of the wrapper component, adjacent the absorbent article 10. When folded, the free end 54 of the packing component is no longer at the opening of the wrapper component. Instead, an apparent free end 54′ is formed, which is the end of the second panel 66. FIG. 9B shows a front view of the closed wrapper component. By having the second side 212 of the wrapper component exposed at the end of the second panel, the different attributes associated with the second side 212 provide a sensory cue to the user as to the location of the opening of the wrapper component 11. In essence, the difference in the attributes of the first side and the second side of the wrapper component provides a sensory cue. As is stated above, the second side 212 of the wrapper component has different attributes from the first side 211, such as, for example color, bond pattern, embossing and the like. Many different combinations of the attributes of the two sides of the two-sided material may be used. Examples of some possible combinations include, for example, different colors on each side, one side is embossed and the other side is not, both sides are embossed with different patterns, etc. In addition, the different attributes can also be a combination of different attributes.

In the present invention, when the free end 54 is turned upward to give an apparent free end 54′, other advantages are obtained. For example, when the free end 54 is folded upward over the second panel 66, the material at the opening becomes thicker, and may be easier for a user to grab the second panel and open the wrapper component. Also, the free end 54 may be further shaped so that the free end has a distinct shape as is discussed above. In addition, the apparent free end 54′, could also be shaped. For example, the free edge 54, having a right side 54R and a left side 54L may be folded from a point on each side of the free edge near the edges 68 and 70 towards the second panel creating a configuration shown in FIG. 9C, where the free edge 54R and the free edge 54L are adjacent each other. As a result, the sides of the second panel 68 and 70 have a new position 68′ and 70′, and create the free edge 54 while the free edges 54R and 54L are shown to be in contact with each other in FIG. 9C, it is also within the scope of this aspect of the invention that, when folded, the edges 54L and 54R are spaced apart with some of the first side of the wrapper component being exposed. As a result of folding the edges 54R and 54L, a shaped second panel is created, as shown in FIG. 9C. One convenient shape which may be created is the V-shaped second panel, with its advantage discussed above. Other shapes could be created by using a combination of different folds and possible cutting of the pouch material.

One advantage of having the free edge of the second panel folded upward, as is shown in FIG. 9A-C is that the wrapper may be open by grapping apparent free edge 54′ or the free edge 54. Unlike prior opening cues which include a single strip of a different color along the free edge, which does not clearly indicate where to grab the second panel to open the wrapper and does not clearly indicate the location of the lone free edge of one side of the color strip, by folding the free edge over the second panel material, the wrapper may be opened by grabbing the free edge 54 or the apparent free edge 54′. This will avoid confusing the user as to the location of the opening. This can be especially helpful to a user in locating the second panel on the wrapper and opening, especially in poor lighting conditions or aiding those users with poor eyesight. The region 213 of the second panel where the second side 212 of the material is visible, will also provide a place where a user a thicker piece of material to grab, thereby making it easier to open the wrapper component. Generally, region 213 will have a length Ls. Generally, Ls will be about the average length of the end section of a human finger of the user group, which is generally in the range of about 20 to 35 mm in length.

In other embodiments (not shown), the sensory cue could include a colored or patterned strip or wave formed on one or both of the second panel and first panel. If a colored and/or patterned strip or wave is used, the color or pattern could be a color or pattern generally not associated with the absorbent article, which may draw the users attention to the second panel 66 and opening associated with the second panel. For example, in the case of feminine care absorbent articles, the color could be a vibrant color such as orange. The sensory cue could further include additional, separate pieces of material applied to one or both of the second panel and first panel. In addition, the second portion of the first panel and the second panel could be made identical (same pattern and/or color), but with the first portion of the first panel being a different color or pattern that is visible through the second panel so as to provide a sensory cue. For example, in one embodiment, the sensory cue is configured by way of a colored adhesive, which is used to adhere the second panel to the first panel. The adhesive is visible through the second panel, which may be relatively see-through.

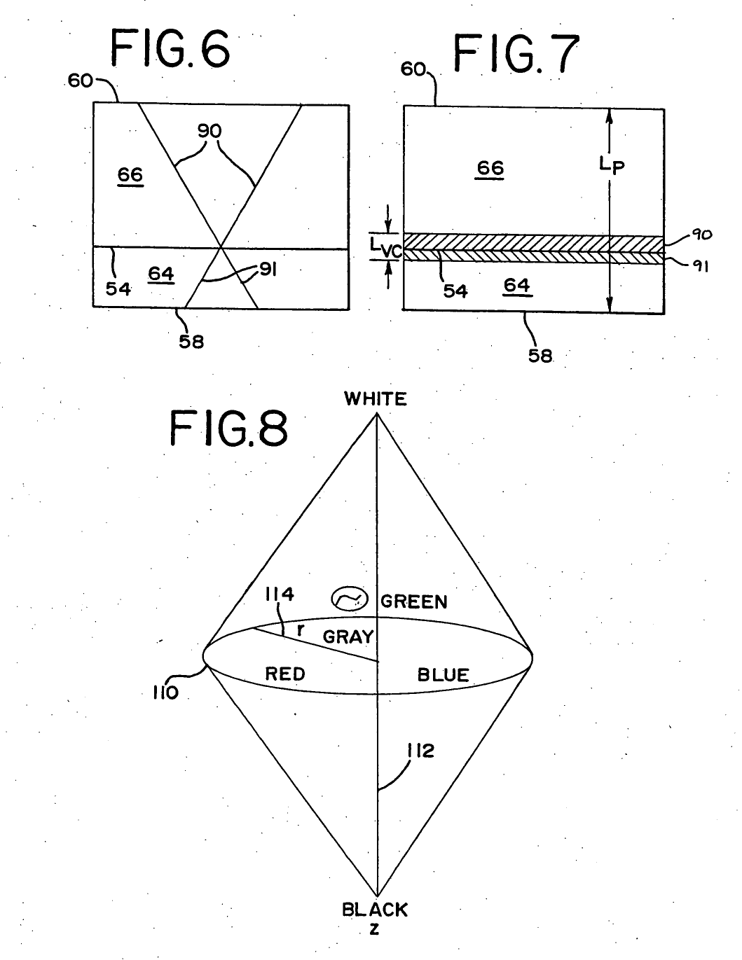

The sensory cue is not limited to only the second panel 66 or the first panel portion 64. Both can have a sensory cue. Referring to FIG. 6, both the second panel 66 has a first sensory attribute 90 and the panel portion 64 has a second attribute cue 91. Both of the first and second sensory attributes 90, 91 create a sensory cue which is directed at and terminates at the free edge 54. In another aspect of the present invention, the sensory cue may be provided on an element of the absorbent article and a clear material may be used as the wrapper component. For example, the sensory cue could be provided on the peel strip or the backsheet of the absorbent article, or on any other portion of the absorbent article, provided that the sensory cue provides indicia as to the location of the opening of the wrapper component.

In yet another embodiment, shown in FIGS. 5A and 5B, a wrapper component web 78 of material is provided with a color gradient, or fades, in a cross-direction 79 from a first side 75, where the material is darker (D) to a second side 73, where the material is lighter (L). As the web of material 78 is moved in the machine direction 77, a folded product component, e.g., a tri-folded feminine pad, is applied to the web of material 78. The web 78 is then folded around the product component, with the first side 75 forming the exterior free edge 54 that overlies the first panel and is spaced from the second side 73, which forms the interior free edge 56. Alternatively, the light side forms the exterior free edge, with the dark side forming the interior free edge. Cross-direction 79 side seals are then successively applied to the folded web, and the web is then cut at the side seals to form a plurality of discrete personal care products.

The color gradient provides a processing advantage in that the contrast between the dark and light ends 75, 73 provides a sensory cue, but without concern about where the fold line 60 is formed. In this way, the color gradient provides a tolerance for forming the fold line 60, in contrast with embodiments where a color or pattern ends at the fold line. In addition to gradient color, a gradient embossing pattern may also be used.

In another embodiment, the second panel 66 and the first panel 64 are each of one color, which when overlapped form a third color. The sensory cue can include a line or intermittent pattern disposed on the second panel or first panel adjacent the free edge. A pattern forming the sensory cue can extend along a portion of the length of the second panel or first panel, along the entirety thereof, and can vary across the width thereof. Of course, it should be understood that the sensory cue can take many forms, and can include various combinations of colors, patterns, shading etc.

In another embodiment, the first panel and second panel could have the same color and/or pattern, but with the second panel being at least partly see-through such that the combined thickness of the first portion and portion of the second panel overlying the first portion has a different appearance than the second portion. For example, the combined thickness of the portion of the second panel overlying the first portion and the first portion may appear darker than the second portion of the first panel, thereby providing a sensory cue directed at the free edge.

In another embodiment, the attributes of the second panel and/or the first panel may be modified by using different materials for the first panel and the second panel by attaching a different material to the wrapper material or a material that is the same as the wrapper material but having a different attribute, such as color, shape, patterns and the like. In one particular embodiment, the material attached is attached to the second panel and the material has a V-shape, described above. By attaching a piece of material to the second panel having a V-shape or other shapes, problems associated with forming a shaped second panel may be alleviated. Other methods include using different materials for the second panel and the first panel. If the second panel is a separate and a distinct material, then the top edge along with side edges would need to be bonded together. In yet another embodiment, the back panel, first panel and the second panel could each be prepared from separate materials.

In some embodiments, the sensory cue includes one or both of a visual cue and a tactile cue. For example, the second panel can have a different texture than the underlying panel, so as to form a tactile cue. In some embodiments, the tactile cue is formed by an embossment, or different embossments. Similarly, a separate piece of material applied to one or both of the second panel or first panel may have a different texture or feel than the other thereof, thereby providing a tactile cue as to the location of the free edge.

In one embodiment, the sensory cue is located on the first panel and is directed at the free edge so as to provide indicia to the user about where their hand should be located such that it can be inserted to lift the second panel. In another embodiment, the sensory cue is located on the second panel opposite where the hand should be located. In either case, the sensory cue provides indicia of the location of the free edge, but also indicia of where the user's hand should be located.

In an alternative embodiment, shown in FIG. 7, the sensory attributes 90 and 91, each is a different color as shown, are disposed on both sides of the free edge 54. In this embodiment, the sensory attributes on the second panel 66 and the first panel 64 terminate on each side of the free edge 54. As a result, unlike FIGS. 10A and 10B, the free edge 54 is readily apparent to the user, and is located at the intersection of the sensory attributes 90 and 91. As a result, a sensory cue is provided. In one embodiment, the length of the sensory cue (Lvc) is less than about 33% of the overall length (Lp) of the product, with the free edge 54 disposed within the coverage of the sensory cue. In other embodiments, Lvc is less than about 25% of Lp, and more desirably less than about 10% of Lp.

Referring to FIGS. 2A and 2C and 3A-3J, in operation, the user locates the free edge 54 of the wrapper component 50 using the sensory cue (visual or tactile) cue 90. The user then grasp the free edge 54 of the second panel 66 and opens the wrapper component to access the personal care product component. The user can then remove the product component and discard the wrapper component.

In another aspect of the present invention, the sensory attributes may be selected in such a way as to aid the user by communicating the direction the free edge should be moved in order to open the wrapper component. In this regard, many techniques may be applied to the second panel or the first panel to help direct the user the direction in which the free edge should be manipulated in order to open the wrapper. Some of those methods are described above in the description of FIGS. 3C, 3D, 3I, 3J, and 6. As is described above, the shape of an envelope relates to the life experiences of opening both sealed and unsealed envelopes by raising the second panel from the point of the second panel. Other methods can be used as well. One method is to provide some depth to wrapper component. In this regard, reference is made to again to FIGS. 3A, 3E and 3F. To provide depth, the pattern of sensory attribute 90 on the second panel 66 shown on FIG. 3A could be raised pattern. When the pattern is raised, second panel 66 is perceived by a user to be in front of the first panel 64. As a result, the sensory attribute 90 not only provides a sensory cue as to the location of the free edge 54; the sensory attribute also conveys that the second panel 66 is in front of the first panel 64. Armed with this information, the user is directed to open the wrapper component by moving the second panel 66 away from the first panel 64. In a similar manner, a recess pattern on the first panel 64 and an unpatterned second panel 66, similar to that shown in FIG. 3E, will convey to the user that the first panel 64 is below or behind the second panel 66. Other techniques include having a recessed pattern on the first panel 64 and a raised pattern on the second panel 66, as may be shown in FIG. 3F. In addition, having an embossed pattern on the second panel 66 and no embossing of the first panel 64, results in the first panel 64 having a smooth surface. Generally, when confronted with a rough (non-smooth) surface and a smooth surface, which results form embossing the second panel 66 and not the first, as shown in FIG. 3A, a user will tend to slide their hand along a smooth surface of the first panel 66, to locate the free edge 54, rather than slide their hand along a non-smooth surface. As a result, the smooth surface and the embossed surface serves to convey to a user how to open the wrapper component.

Depth and providing a sensory cue to the user of how to open the wrapper component may also be accomplished using color. Attention is again directed to FIG. 3B. By providing the second panel 66 with a warm color (e.g., red, yellow, orange) and the second panel with a cool color (blue, purple or green), a user will perceive that the second panel 66 is in front of the first panel 64. Again, if the second panel 66 is perceived by the user to be in front of the first panel 64, the user is provided with a sensory cue how to open the wrapper component.

Other techniques may also be used to convey to the user how to open the wrapper. One example of this is provided in FIG. 11. FIG. 11 shows a first panel 64 with a sensory attribute 90, which terminates at the free edge 54 of the second panel 66. The sensory attribute has a familiar shape in which appears to be partially blocked out. One shape which is of particular is a semi-circle; however, other shapes may be used. Generally the shape is prepared from a color which is darker or cooler that than the color of the second panel 66 or the remainder of the first panel, providing a shadow-like effect. The color can be provided on the first panel using any of the techniques describe above. As a result, of the combination of sensory attributes 90, color and the semi-circle shape, a sensory cue is provided to the user, wherein the sensory cue identifies the free edge and provides information to the user of how to open the wrapper component.

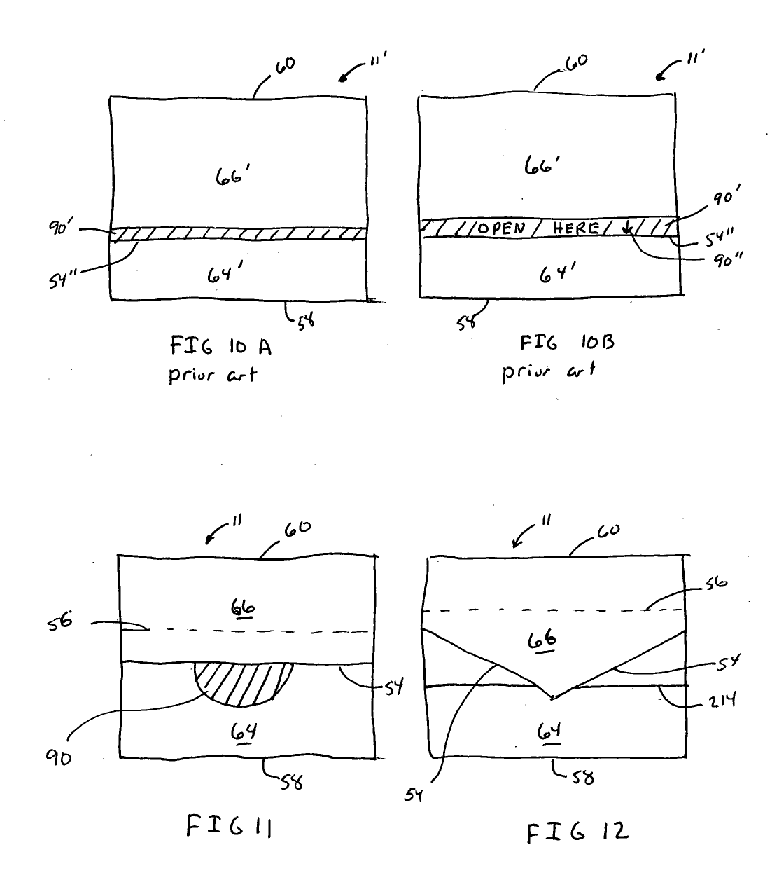

FIG. 12 shows yet another technique to show how to open and locate free edge 54 of the wrapper component. The technique shown in FIG. 12 is called interposition. Interposition is term used to describe the sensory cue for the discrimination of the relative distances of two objects that results when one object partially obscures or overlaps the outline of another objects. In FIG. 12, a line 214 is printed or otherwise provided on the first panel 64 and the second panel 66 overlaps the line 214 in such a way that the line appears behind the second panel 66. As a result, a sensory cue is provided to a user who will identify the second panel 66 and the free edge 54 as being in front of the line 214. Stated another way a user perceives the second panel being closer than the line 214. As a result, the second panel 66 appears to be in front of the first panel 64.

The absorbent article may be placed in the wrapper component in a number of different ways. The absorbent article may be folded and placed on an unfolded wrapper material and the wrapper component is formed by folding the wrapper material into a pouch-like configuration around the absorbent article. In other methods, such as those described above, the wrapper and pouch material may be folded together. The wrapper material and the absorbent article may be aligned in a machine direction, and folded simultaneously. Other methods which can be used to form the wrapper/absorbent article combination include, for example, placing the absorbent article across the wrapper material such that one of the wrapper material or the absorbent article is aligned in the machine direction and the other is aligned in the cross machine direction, folding the absorbent article on the wrapper and folding the wrapper around the absorbent article.

In the above description, it is generally stated or implied that there is a single absorbent article within the wrapper component. However, it is within the scope of the present invention that a plurality of absorbent articles may be present in each wrapper component of the present invention.

To open the wrapper component of the present invention to access the personal care product placed in a wrapper component, the sensory cue provided on the wrapper is used to locate the free edge of the second panel; and the second panel is manipulated is such a way that the wrapper component is opened and the personal care product component disposed within the wrapper is removable from the wrapper. Manipulating the second panel generally includes grasping the second panel by a user's fingers and pulling the second panel upwards away from the first panel. In some configurations, such a second panel with perforations, the second panel may be torn away from the side seams of the wrapper component.

Other embodiments not specifically discussed above may also be used to provide a sensory cue to the user as to the location of the free edge. For example, the sensory cue could be provided on the absorbent article, for example on the baffle or garment facing side of the absorbent article. This would allow a clear wrapper to be used. In addition, the free edge of the wrapper could be located at the top 60 or bottom 58 of the wrapper component.

Although the present invention has been described with reference to various embodiments, those skilled in the art will recognize that changes may be made in form and detail without departing from the spirit and scope of the invention. As such, it is intended that the foregoing detailed description be regarded as illustrative rather than limiting and that it is the appended claims, including all equivalents thereof, which are intended to define the scope of the invention.

1. A personal care product comprising:
at least one wrapper with at least one free edge;
at least one personal care article disposed within the wrapper; and
at least one sensory cue providing clear indicia as to the location of at least one free edge of the wrapper.

2. The personal care product of claim 1, wherein
the wrapper comprises a wrapper component comprising a first panel, a back panel and a second panel, said first panel is connected to said back panel and said second panel is connected to said back panel at an opposite end from where the first panel is connected to the back panel, said second panel having a free edge positioned adjacent said first panel, wherein the second panel comprises a first set sensory attributes which terminate at the free edge of the second panel and the first panel comprises a second set of sensory attributes which are adjacent the free edge of the second panel, wherein at least one attribute from the first set of sensory attributes or at least one attribute from the second set of attributes is different from attributes from the other set, wherein the different attribute provides a contrast between the second panel and the first panel; the contrast provides a sensory cue providing clear indicia as to the location of the free edge of the second panel.

3. The personal care product of claim 2, wherein the sensory cue comprises a visual cue, a tactile cue or a combination of a visual cue and a tactile cue.

4. The personal care product of claim 2, wherein the first set of sensory attributes comprises a first color, a first printed pattern or first embossed pattern, and the second set of sensory attributes comprise second color, a second printed pattern or second embossed pattern.

5. The personal care product of claim 2, wherein said free edge overlies at least a portion of said first panel.

6. The personal care product of claim 4, wherein said first set of sensory attributes comprises a first color and the second set of sensory attributes comprise a second color different than said first color.

7. The personal care product of claim 2, wherein at least one of the first set of sensory attributes and the second set of sensory attributes comprise an embossed or printed pattern.

8. The personal care product of claim 7, wherein the first set of sensory attributes comprises a first embossed or printed pattern, and the second set of sensory attribute comprises a second embossed or printed pattern, and the second pattern is a different pattern from said first pattern.

9. The personal care product of claim 7, wherein only one of the first set of sensory attributes and the second set of sensory attributes comprise an embossed or printed pattern, and one other set of sensory attributes is devoid of an embossed or printed pattern.

10. The personal care product of claim 8, wherein each pattern is printed.

11. The personal care product of claim 9, wherein the pattern is printed.

12. The personal care product of claim 8, wherein each pattern is embossed.

13. The personal care product of claim 9, wherein the pattern is embossed.

14. The personal care product of claim 2, wherein said at least one of the first set of sensory attributes and the second set of sensory attributes comprise an arrow shape directed at said free edge.

15. The personal care product of claim 2, wherein the first set of attributes comprises the free edge of the second panel comprising a non-linear shape.

16. The personal care product of claim 15, wherein the non-linear shape comprises a V-shape, a wave or a scalloped shape.

17. The personal care product of claim 16, further comprising at least one additional attribute which is different between the first set of attributes and the second set of attributes.

18. The personal care product of claim 2, wherein the second panel comprises a first material and the first panel comprises a second material, wherein the first material is different from the second material.

19. The personal care product of claim 2, wherein the wrapper comprises a material having a first side and a second side, each having sensory attributes, at least one sensory attribute of the first side are different than the second side and first side comprises an outer surface of the wrapper.

20. The personal care product of claim 19, wherein the free edge of the second panel is folded over the second panel such that a portion of the outer surface of the second panel is adjacent itself and exposing the second side wrapper component and the second side of the wrapper component contrasts the first side of the wrapper present on an outer surface of the first panel of the wrapper.

21. The personal care product of claim 20, wherein the second panel is folded in a manner which provides a shape to the portion of the second panel adjacent the panel has a shape other than a straight line.

22. The personal care product of claim 21, wherein the shape comprises a V-shape.

23. The personal care product of claim 2, wherein the sensory cue also provides indicia to a user of the personal care product of how to open the wrapper component.

24. The personal care product of claim 14, wherein the sensory cue also provides indicia to a user of the personal care product of how to open the wrapper component.

25. The personal care product of claim 2, wherein the second panel comprises a flap.

26. The personal care product of claim 2, further comprising at least one additional panel.

27. The personal care product of claim 2, wherein one of the sensory attributes comprises a gradient color or a gradient embossing.

28. A personal care product comprising:
at least one wrapper with at least one free edge;
at least one personal care article disposed within the wrapper; and
at least one sensory cue providing clear indicia as to how to open the wrapper.

29. The personal care product of claim 28, wherein the sensory cue also provides clear indicia as to the location of the free edge.

30. A method of using a personal care product comprising:
providing a personal care article disposed in a wrapper component, said wrapper component having a free edge and sensory cue providing clear indicia as to the location of the free edge of the wrapper.
locating said free edge using said sensory cue; and
manipulating said second panel and opening said wrapper component to access said personal care product component.

31. The method of claim 30, wherein the wrapper component comprises a first panel, a back panel and a second panel, said first panel is connected to said back panel and said second panel is connected to said back panel at an opposite end from where the first panel is connected to the back panel, said second panel having a free edge positioned adjacent said first panel, wherein the second panel comprises a first set of sensory attributes which terminate at the free edge of the second panel and the first panel comprises a second set of sensory attributes which are adjacent the free edge of the second panel, wherein at least one attribute from the first set of sensory attributes or at least one attribute from the second set of attributes is different from attributes from the other set, wherein the different attribute provides a contrast between the second panel and the first panel; the contrast provides the sensory cue providing clear indicia as to the location of the free edge of the second panel.

32. The method of claim 31, wherein said product component comprises an absorbent product component.

33. The method of claim 32, wherein said second panel comprises a different sensory attribute and the sensory cue is a visual cue.

34. The method of claim 32, wherein said first panel comprises a different sensory attribute and the sensory cue is a visual cue.

35. The method of claim 32, wherein said first set of sensory attributes comprises a first color and the second set of sensory attributes comprise a second color different than said first color.

36. The method of claim 32, wherein the first set of sensory attributes comprises a first embossed or printed pattern, and the second set of sensory attributes comprises a second embossed or printed pattern, and the second pattern is a different pattern from said first pattern.

37. The method of claim 32, wherein said at least one of the first set of sensory attributes and the second set of sensory attributes comprises an arrow shape directed at said free edge.

38. The method of claim 32, wherein the wrapper comprises a material having a first side and a second side, each having sensory attributes, at least one sensory attribute of the first side are different than the second side and first side comprise an outer surface of the wrapper.

39. The method of claim 38, wherein the free edge of the second panel is folded over the second panel such that a portion of the outer surface of the second panel is adjacent itself and exposing the second side wrapper component and the second side of the wrapper component contrasts the first side of the wrapper present on an outer surface of the first panel of the wrapper.

40. The method of claim 31, wherein said manipulating comprises grasping and pulling the second panel.
